(19) United States
(12) Patent Application Publication
(10) Pub. No.: US 2013/0096532 A1
(43) Pub. Date: Apr. 18, 2013

(54) Polymer-Based Micro-Needle Array Designs, Fabrication Processes, and Methods of Use Thereof for Drug Delivery

(71) Applicant: Rutgers, The State University of New Jersey, New Brunswick, NJ (US)

(72) Inventors: Tugrul Ozel, Bedminster, NJ (US); Thanongsak Thepshonti, Piscataway, NJ (US); Nicola Milesi, Salo (IT)

(73) Assignee: RUTGERS, THE STATE UNIVERSITY OF NEW JERSEY, New Brunswick, NJ (US)

(21) Appl. No.: 13/654,324

(22) Filed: Oct. 17, 2012

Related U.S. Application Data
(60) Provisional application No. 61/547,975, filed on Oct. 17, 2011.

(51) Int. Cl. A61M 37/00; B23C 5/00; B23C 3/00

(52) U.S. Cl. CPC A61M 37/0015; B23C 3/00; B23C 5/006
USPC 604/506; 604/173; 409/131; 407/119

(57) ABSTRACT

A micro-needle array is provided that may be used to deliver a bioactive agent to a therapeutic target. The micro-needle array preferably includes a substrate, a plurality of micro-needles integral with the substrate, and a bioactive agent. At least one micro-needle preferably includes a top surface, a bottom surface, a side surface, and a cavity defined by an inner surface. The bioactive agent may be disposed on the substrate and the plurality of micro-needles. The at least one micro-needle may further include a slit connecting the cavity to an aperture, the slit extending from the top surface to the bottom surface. A plurality of micro-needles on a patch is also disclosed for transdermal drug delivery applications.

This application claims priority to U.S. Provisional Application No. 61/547,975 filed Oct. 17, 2011.

### FIELD OF THE INVENTION

This invention relates to the fields of drug delivery and micro-needle array fabrication and use.

### BACKGROUND OF THE INVENTION

Several publications and patent documents are cited through the specification in order to describe the state of the art to which this invention pertains. Each of these citations is incorporated herein by reference as though set forth in full.

Human skin is composed of three primary layers; i) the epidermis, which provides waterproofing and serves as a barrier to infection; ii) the dermis, which serves as a location for the appendages of skin; and iii) the hypodermis (subcutaneous adipose layer). Epidermis is the outermost layer of the skin. It forms the waterproof, protective wrap over the body's surface and is made up of stratified squamous epithelium with an underlying basal lamina. The epidermis contains no blood vessels, and cells in the deepest layers are nourished by diffusion from blood capillaries extending to the upper layers of the dermis. The outer layer known as the stratum corneum, is 10-15 μm thick and is primarily made of dead tissue (see FIG. 1). This layer is often the primary barrier to fluid transport to the body. Viable epidermis, up to 50-100 μm below the stratum corneum, contains living cells, but is devoid of blood vessels and contains few nerves. Underneath, dermis comprises the bulk of the skin volume and contains nerves and blood vessels [2].

It is an object of the invention to provide painless micro-needle patches comprising needles so small and short that they cannot reach the dermis and thereby avoid activating receptors for pain.

### SUMMARY OF THE INVENTION

In accordance with the present invention, a patch comprising a plurality micro-needles is provided which is particularly useful for the minimally invasive sampling of a biological fluid and/or the minimally invasive delivery of a drug or other formulation across the skin. In one embodiment, the plurality of micro needles are produced using polymers and have solid structures comprising a base at the proximal end and vertex at the distal end to which the therapeutic/bioactive agent may be applied. In another embodiment, the micro-needle(s) on the patch has a structure having a base at a proximal end and a vertex at a distal end, and an open lumen extending there through and through which fluid may be transferred. The structure defines a structural axis that intersects the lumenal axis defined by the open lumen. The point of intersection between these axes is at a point below the vertex of the micro-needle to provide a sharp apex at the distal end of the micro-needle and defines the general configuration of the distal end of the micro-needle, which may be selected or customized depending on the intended use of the microneedle. In preferred embodiments, the length of the micro-needle is between about 0.1-1 mm and the diameter is about 10-100 μm. According to the shape and the purpose, micro-needles can be fabricated in metals, silicon or silicon dioxide, polymers and glass. Moreover they offer a broad range of advantages when compared with traditional hypodermic needles. In a particularly preferred embodiment the patch is made of a polymer and comprises biodegradable micro-needles.

The invention also provides a micro tool for use in the fabrication process described herein.

Methods of making and using the micro-needle of the present invention as well as kits comprising one or more of the micro-needles are also provided. In a preferred embodiment, methods of fabrication employ a micro-milling process that produces a superior burr free micro needle of uniform shape and configuration.

### BRIEF DESCRIPTION OF THE DRAWINGS

FIG. 1: Cross section of human skin.

FIG. 2: Micro-needle patch prototype on polymer.

FIG. 3: Various shapes (tip, oblique, and side) for micro-needle tips.

FIG. 4a: Molding structure. FIG. 4b: First exposure process. FIG. 4c: Second exposure process. FIG. 4d: Second coating process. FIG. 4e: Final structure.

FIG. 5: Multi-layer structure of multiple-layer fluidic structure. Inset provides close-up view.

FIG. 6: Solid micro-needle array model. FIG. 6a: pyramidal design. FIG. 6b: conical design. FIG. 6c: dwarf conical design.

FIG. 7: Hollow micro-needle array models. FIG. 7a: shell type hollow structure. FIG. 7b: design of holes in the middle of the micro-needle with cross-sectional views. FIG. 7c: design of holes parallel to the side of the micro-needle with cross-sectional views. FIG. 7d: design of straight holes to the right of the micro-needle with cross-sectional views.

FIG. 8: Geometrical parameters. h: needle height; a: horizontal space; b: vertical space.

FIG. 9: Tool dimensions and characteristic. Schematics and images of the tool and tool tip are provided along with dimensions of the tip.

FIG. 10: Experimental setup for micro-milling of polymers.

FIG. 11: “Grill” tool path strategy. FIG. 11A: zig-zag path with a stepover distance. FIG. 11B: deeper cut. FIG. 11C: deeper cut. FIG. 11D: schematic and image of final product.

FIG. 12: “S” tool path strategy. FIG. 12A: “S” path. FIG. 12B: “S” path in the transversal direction. FIG. 12C: schematic and images of final product.

FIG. 13: Customized tool path strategy for conical micro-needles. FIG. 13A: initial pattern. FIG. 13B: deeper cut. FIG. 13C: deeper cut. FIG. 13D: final passes of tool. FIG. 13E: schematic and image of final product.

FIG. 14: Customized tool path strategy for conical micro-needles. FIG. 14A: roughing step. FIG. 14B: circular cutting pattern. FIG. 14C: deeper cut. FIG. 14D: deeper cut. FIG. 14E: schematic and image of final product.

FIG. 15: Square based pyramidal shaped micro-needles including side view, view from below, and close-up view.

FIG. 16: Circular based pyramidal shaped micro-needles including side view and view from below.

FIG. 17: Work-piece with 1 mm and 2 mm micro-needle array viewed from two different angles.

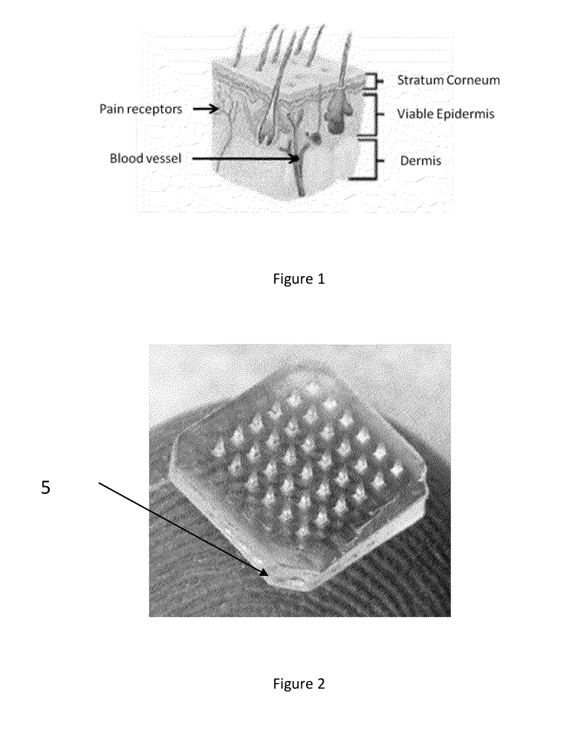

### DETAILED DESCRIPTION OF THE INVENTION

Micro-needles are new medical devices with the same purpose of classic hypodermic needles but fabricated on micro-scale often in the form of arrays in various materials. During the past few decades scientists and engineers have spent considerable amount of time and resources to develop micro-needle patches for drug delivery. These devices aim to replace the hypodermic needles and consist of a patch with micro-sized needles. Micro-needle patches are about the size of a postage stamp and hold hundreds of micro-sized needles, each less than a millimeter long and in different shapes. These patches generally do not induce pain since these micro-sized needles penetrate into the skin small enough and do not reach pain receptors and they can be applied without the help of a health professional [1].

### Definitions:

The term “biocompatible,” as used herein, refers to a material that is substantially non-toxic in the in vivo environment of its intended use, and that is not substantially rejected by the patient's physiological system. A biocompatible structure or material, when introduced into a majority of patients, will not cause an undesirably adverse, long-lived or escalating biological reaction or response. Such a response is distinguished from a mild, transient inflammation which typically accompanies surgery or implantation of foreign objects into a living organism.

The term “biodegradable,” as used herein, refers to a material that dissipates upon implantation within a body, independent of the mechanisms by which dissipation can occur, such as dissolution, degradation, absorption and excretion. The actual choice of which type of materials to use may readily be made by one of ordinary skill in the art. Such materials are often referred to by different terms in the art, such as “bioresorbable,” “bioabsorbable,” or “biodegradable,” depending upon the mechanism by which the material dissipates. The prefix “bio” indicates that the erosion occurs under physiological conditions, as opposed to other erosion processes, caused for example, by high temperature, strong acids or bases, UV light or weather conditions.

The term “controlled release,” as used herein, refers to the release of an agent at a predetermined rate. A controlled release may be constant or vary with time. A controlled release may be characterized by a drug elution profile, which shows the measured rate that the agent is removed from a device in a given solvent environment as a function of time. For example, a controlled release elution profile from a medical device may include an initial burst release associated with the deployment of the device, followed by a more gradual subsequent release. A controlled release may be a gradient release in which the concentration of the agent released varies over time or a steady state release in which the agent is released in equal amounts over a certain period of time (with or without an initial burst release).

The term “barrier layer,” as used herein, is any layer that is placed over at least a portion of a bioactive agent present in or on a portion of a device of the present invention. In general, the bioactive agent will not be present in the barrier layer. Any mixing of a bioactive agent with the barrier layer is unintentional and merely incidental. The barrier layer may or may not be the outer-most layer present on the device. For example, a bioactive agent may be coated onto a surface of the device, a first barrier layer placed over the bioactive agent and further barrier layers and layers containing the same or a different bioactive agent placed on the first barrier layer. The barrier layer may control the release of the bioactive agent from the device upon implantation.

The term “carrier material,” as used herein, refers to a material that forms a mixture with bioactive agent on or in a device of the present disclosure. The carrier material may control the release of the bioactive agent from the device.

The term “bioactive agent,” as used herein, refers to any pharmaceutically active agent that produces an intended therapeutic effect on the body to treat or prevent conditions or diseases.

The term “treatment” or “treating,” as used herein, describes the management and care of a human or veterinary patient for the purpose of combating or preventing a disease, condition, or disorder and includes the administration of a bioactive agent to alleviate the symptoms or complications, or eliminate the disease, condition, or disorder.

The term “therapeutically-effective amount,” as used herein, is the minimal amount of a bioactive agent which is necessary to impart therapeutic benefit to a human or veterinary patient. For example, a “therapeutically effective amount” to a human or veterinary patient is such an amount which induces, ameliorates or otherwise causes an improvement in the pathological symptoms, disease progression or physiological conditions associated with or resistance to succumbing to a disorder.

### Advantages of Micro-Needles

As explained above, the dimensions of the micro-needles can be optimized to avoid contact with the dermis layer of the skin yet still deliver effective amounts of bioactive agents. This approach prevents the needles from activating pain receptors, thus the patient only experiences the sensation of being touched by a flat patch. This leads to other advantages; indeed having a painless vaccination means that less people avoid vaccination. Moreover the dimensions enable preparation of the micro-needles in a patch for simple administration, possibly by patients themselves and can be inserted painlessly onto the skin without specialized training. These micron-scale needles can be mass produced using low-cost methods for distribution to doctors' office, pharmacies and, possibly, people's homes. In addition, having such small needles that most of time could dissolve through the skin would eliminate the risk of sharp hazardous waste and reduce the possibility of undesirable re-use of the device.

Other advantages of the micro-needle patches could include lower dosage requirements. Lower doses could be particularly important because flu vaccine production capacity sometimes is limited for seasonal vaccine, and a future influenza pandemic would require much greater production of vaccine. Replacing a hypodermic needle with a micro-needle patch also could significantly impact the way other vaccines are delivered, and could be particularly beneficial in developing countries.

In summary, the advantages of micro-needles over conventional needles include; i) no painful piercing of the skin, ii) self-application, easily distributed and sold over the counter, iii) no sharp hazardous waste after immunization, iv) more effective vaccinations, and v) more cost effective than hypodermic needles.

Micro-Needle Types and Fabrication Methods

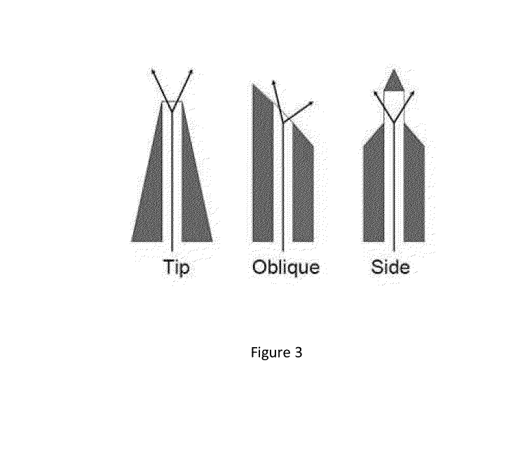

With the goal of decreasing the pain associated with drug injection and blood extraction researchers have investigated transdermal micro-needle array technologies. Several different types of micro-needles have been fabricated. It is possible to find solid (straight, bent, filtered) and hollow micro-needles. Solid needles could be used to increase drug diffusion rate by using a drug patch. Hollow needles which include tapered and beveled tips (see FIG. 3) allow delivery of microliter quantities of drugs to specific locations in human body. Micro-needles arrays of hollow needles can also be used together with a pump system to remove fluid from the body for analysis such as blood glucose measurements. Furthermore, small enough micro-needles could even provide drug administration to individual cells and also open possibilities for stem cell research.

The first micro-needle arrays reported in the literature were etched into a silicon wafer and developed for intracellular delivery in vitro by Hashmi et al. [5]. These needles were inserted into cells and nematodes to increase molecular uptake and gene transfection. Henry et al. [1] conducted the first study to determine if micro-needles could be used to increase transdermal drug delivery. An array of solid micro-needles was embedded in cadaver skin, which exhibited skin permeability to a small model compound.

The development of micro-needles began in the late 1990s with micro-fabrication of solid needles made from silicon, using microlithography and etching technologies originally developed for the microelectronics industry. This method produced arrays of up to 400 needles designed to punch holes in the outer layer of skin to increase its permeability to small molecules applied with the patches. That work has broadened to include both solid and hollow micro-needles. These creations range in size from 1 millimeter in length to only 25 μm in diameter [3].

Kaushik et al. [6] carried out a small trial to determine if micro-needles are perceived as painless by human subjects. Micro-needle arrays were inserted into the skin of twelve subjects and compared to pressing a flat surface against the skin and inserting a 26-gauge hypodermic needle into the skin surface. Subjects were unable to distinguish between the painless sensation of the flat surface and the one caused by micro-needles. All subjects found the sensation caused by the hypodermic needle to be much more painful. Other studies have also reported that micro-needles can be applied to human subjects in a painless manner [7, 8].

One drawback reported in the literature was the breakage of metal needles inside the skin since metal micro-needles were not elastic but rather brittle. One solution to this problem is by changing the material from metals to polymers. It was easier, cheaper and faster to obtain the desired shape and dimensions while maintaining strength. Moreover micro-needles were made of a harmless dissolving polymer that was mixed with a freeze-dried vaccine. It means that instead of the needles injecting a fluid, the needles quickly dissolve to become the fluid. This way when the patch is removed there is nothing sharp left on it and it is readily and safely disposable without hazardous outcome.

Biodegradable polymer micro-needles have recently been fabricated and characterized. The advantage of biodegradable polymer needles is that they can be cost-effectively produced and do not pose a problem of breakage in the skin due to biocompatibility and biodegradability [9, 10]. In addition, coated encapsulated molecules within micro-needles can dissolve in the skin and leave no biohazardous waste thereafter [12].

Fabrication of biodegradable polymer micro-needles with sharp, beveled or tapered tips is possible by using masking and etching MEMS processing techniques or within an in situ lens-based lithographic technique.

Presently, many different types of micro-needles can be found in literature: solid, hollow, with different shapes for different purposes, and with different materials. The broad range of sizes, shapes and materials must be assessed and optimized to permit production of micro-needle arrays customized for the type and volume of drug to be delivered, the time period of use, and most importantly, minimizing the pain.

Several micro-electro-mechanical-systems (MEMS) type micro-needle designs have been developed by using semiconductor based fabrication methods. Moving away from the original expensive microelectronics-based fabrication techniques, manufacturing process chains have been developed for low cost production of micro-needles in metal and polymer materials. In these process chains, techniques to fabricate molds are utilized then polymeric micro-needles are fabricated with injection molding. The creation of the molds is done by using a master prototype for micro-needles. Metallic micro-needles can also be produced through electro-deposition and glass micro-needles can be fabricated using glass drawn micropipette techniques as reported in literature [3].

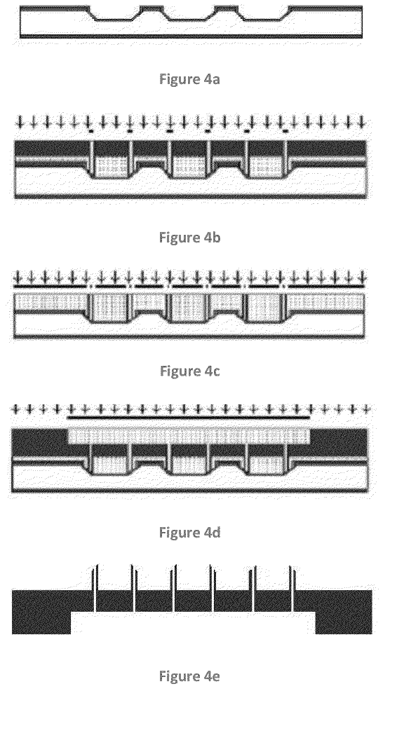

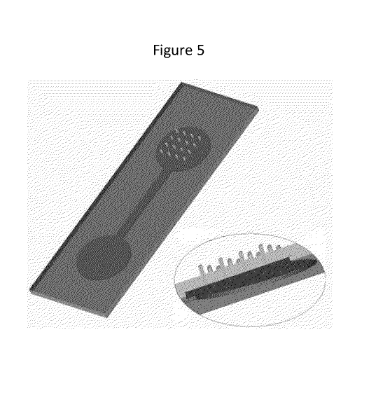

Multi layer structures for polymeric micro-needles are proposed by Kuo & Chou [13]. The fabrication process for the multi layer structure is shown in FIG. 4 and multi-layer structure of multiple-layer fluidic structure is shown in FIG. 5. The first step is the deposition of SiN4 layer on single crystal silicon wafer as mask material with a dry etching process. Then, there are duplicate recesses on the silicon substrate are formed and each recess is shaped into the inclined sidewall structure during the etching process. A release layer and a polymeric layer are coated on top of the structure respectively. Following a multiple exposure process, a multi layer structure is fabricated and a supporting layer of micro-needles is built (FIGS. 4b and 4c). A polymeric layer is coated on the exposed layer and the micro-channels are constructed. As a final step the silicon substrate is peeled off and the micro-needles are developed [13]. According to Kuo & Chuo [13] this technique can fabricate hollow micro-needles with sharp tips and the dimensions of 50 μm and length of 600 μm.

Very small micro-needles could provide highly targeted drug administration to individual cells. These are capable of very accurate dosing, complex release patterns, local delivery and biological drug stability enhancement by storing in a micro volume that can be precisely controlled. These studies suggest that micro-needles may provide a powerful new approach to transdermal drug delivery.

Thus, in accordance with the present invention, micro-needle arrays have been designed and fabricated using a based patch prototype with micro-milling of polymeric materials. The i) design, ii) process planning for prototyping, iii) fabrication, iv) evaluation of results, and v) discussion and conclusions are provided herein. The micro-needle array patches described herein enable cost-effective and less painful approaches for drug delivery (e.g., vaccination) thereby providing an improved drug delivery medical device.

The following example is provided to illustrate certain embodiments of the invention. It is not intended to limit the invention in any way.

### EXAMPLE 1

### Design and Fabrication Process Chains for Polymeric Micro-Needle Arrays

### Design

Basic needle shapes (pyramidal, conical, cylindrical, etc.) and design parameters such as dimensions and micro-needle array density have been investigated. In parallel, a direct low cost and rapid solution to fabricate a micro-needle array manufacturing method is also provided. Machining of a pyramidal shape single micro-needle object using micro-milling was initially investigated. Furthermore fabrication of pyramidal shaped micro-needle array using many individual micro-needles was also performed on a polymer workpiece.

### Selection of Basic Needle Shapes

In the beginning a square shaped pyramidal micro-needle structure typically 1 to 3 mm tall was selected as the basic needle shape. The aspect ratio of these micro-needles was the same of the tool which is about 3. Other shapes including squared pyramidal and circular pyramidal (conical) have been considered. These shapes were considered because they can be fabricated by using single edge, zero helical angle straight but tapered and specially fabricated carbide micro-tools.

### Solid Needle Array Design

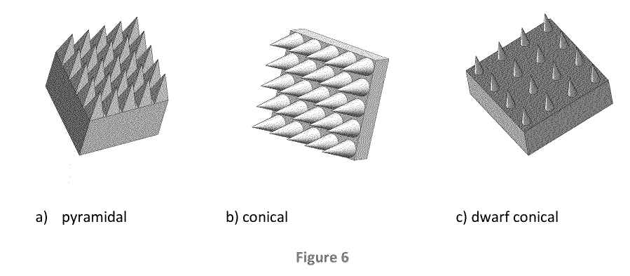

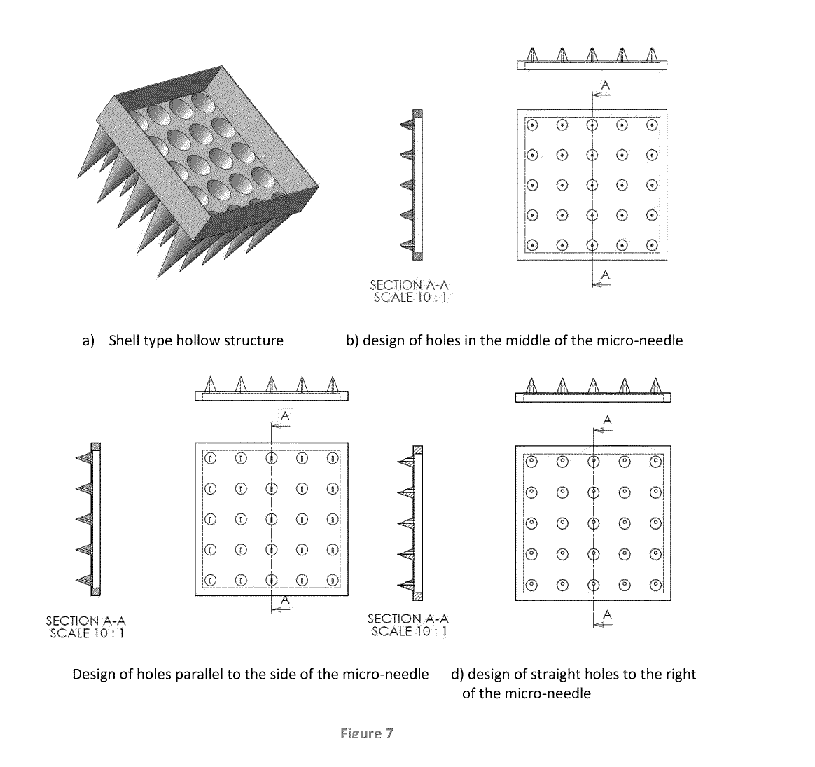

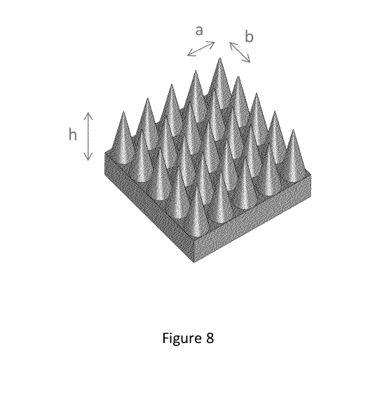

At first, a design with solid micro-needle array type structure was constructed. But also a design with hollow micro-needle array type structure was also considered for further development to store liquid or freeze like substance for vaccination and immunization. Since hollow micro-needle structures are more fragile and delicate to fabricate, a choice of solid micro-needle structure was made for prototyping using micro-milling. Typically in micro-milling tool forces are strong enough to bend, damage and break the fragile and thin micro-structures even if they are made in polymers. For these reasons initial design for basic micro-needle geometry was selected as in the order of millimeters but a further scaling down was planned to keep the micro-needle array structure small enough to be inserted into the human skin and yet not reach to the pain receptors in the Dermis section of the skin tissue. A number of micro-needle array design as solid structures has been performed and these are shown in FIG. 6.

In FIG. 6a, a pyramidal shape design is shown which can be fabricated using a tapered straight cutting edge engraving type micro-milling tool by following linear tool path patterns. In FIG. 6b, a conical solid micro-needle array design is shown which can be fabricated by using the same micro-milling tool but circular tool paths. And finally FIG. 6c, shows a small height, shallow, dwarf type conical solid micro-needle shape design. In this case if the density of the micro-needle array either horizontal or vertical is considered as a design parameter by selecting a shallow design such requirements can be achieved.

### Hollow Needle Array Design

A number of micro-needle array designs incorporating hollow structures have been developed and these are shown in FIG. 7. In FIG. 7a, a modification to the solid needle array design is introduced to create a shell type hollow structure. Specifically, design of holes for delivering liquid drug to be injected has been performed. In FIG. 7b, straight holes in the center of the micro-needles are designed as the most logical first choice. However in the design the sharp tips of the micro-needles are removed. Therefore other alternatives have been sought. One alternative is to fabricate the hole parallel to the side surface of the micro-needle as shown in FIG. 7c. In this case the holes are not straight but angled. This design maintains the sharp tip of the micro-needle. However it creates lower strength for the micro-needle when it is inserted into human skin. The last alternative was to design the holes off-centered in the micro-needles so that the sharp tip of the micro-needle is still maintained while the holes are straight and the hollow structure possesses some strength.

### Design Parameters

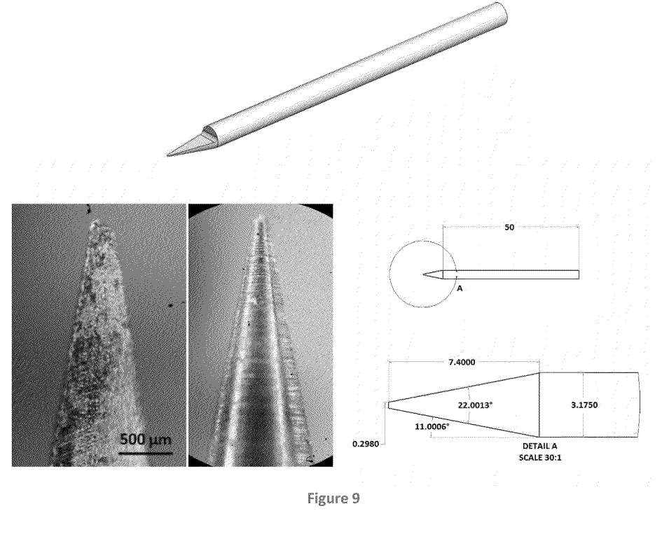

There are a number of design parameters which can be varied when developing micro-needles arrays. These are the basic shape of a micro-needle, the height, the base diameter (conical shape) or the base dimension (square based pyramidal shape), horizontal and vertical spaces, and linear or circular array type. The micro-pyramidal shape array was first defined taking account of the biomedical needs and the milling limits. The arrays had to be made of biocompatible material, the height must be between 0.6 and 1.5 mm and the tip had to be both sharp and resistant to penetrate easily through the skin but not break inside. The micro-milling approach was designed to specifically address these problems, e.g., maintenance of the sharpness and resistance of the tip, but facilitated the ability to obtain specific shapes. Table 1 and FIG. 9 summarize the design parameters.

TABLE 1
Design parameters for the micro-needles

| Variable | Description | Value | |
|---|---|---|---|
| Shape | Cones, Pyramids, etc. | | |
| h | Needle height | 0.6-1.5 | mm |
| d | Base diameter or base dimension | 0.3-0.7 | mm |
| a | Horizontal space | 0.5-2 | mm |
| b | Vertical space | 0.5-2 | mm |
| Array | Linear, Circular patterns, etc . . . | | |
| type | | | |

### Process Planning for Fabrication

In order to fabricate micro-needle arrays, a process plan that includes the selection of the tool geometry and material, the selection of work material, the selection of micro-milling strategy and the parameters was developed and carried out at set forth below.

### Selection of Tool Geometry

The micro-tool was selected as a single flute straight cutting edge tungsten carbide engraving tool. This tool enabled fabrication of micro-needle features as a negative geometry of the tool tip after the material removal process. A tool geometry that is available in an engraving type straight but tapered cutting edge was utilized in fabrication of the micro-needle arrays. The characteristics of the tool geometry and geometrical parameters are shown in FIG. 9 and Table 2. The tool tip had a flat bottom with 0.298 mm width. That assured creating a distance between the base features of the micro-needles. Depending on the axial depth of cut taken during the micro-milling, this distance between the micro-needles within an array can be controlled.

TABLE 2
Geometrical parameters for the micro-tool.

| Shank diameter | 3.1750 mm (⅛ inches) |
|---|---|
| Tip width | 0.2980 mm |
| Tip length | 7.4000 mm |
| Cutter angle | 22.0013° |
| Cutting length | 2.7420 mm |
| Overall tool length | 57.4000 mm |
| Aspect Ratio l/d | 24.8322 |

### Selection of Work Material

There are a number of materials that can be used for fabrication of micro-needles. The first group is polymers which are typically inexpensive, widely available, biocompatible, and possess good machinability such as Acrylic, ABS, PEEK, and PC. In addition, some metal alloys such as stainless steel and titanium alloys which are also biocompatible and possess good machinability and offer high strength and durability can be considered. A summary of the material properties and characteristics that can employed in micro-needle arrays is given in Table 3.

TABLE 3
Material properties and characteristics.

| | | | Thermal | Young | Tensile | | Machinability | | |
|---|---|---|---|---|---|---|---|---|---|
| | Tm | Tg | conductivity | modulus | Strength | | (at room | | |
| Material | [° C.] | [° C.] | [W/m-K] | [GPa] | [MPa] | Cost | temperature) | Biocompatibility | Transparency |
| Acrylic | 230 | 105 | 0.187-0.216 | 2.4-3.3 | 50-80 | Low | Good | Yes | Yes |
| (PMMA) | | | | | | | | | |
| ABS | 105 | 70 | 0.128-0.200 | 2.3-2.9 | 40-60 | Low | Good | Yes | Yes |
| PEEK | 343 | 143 | 0.240-0.300 | 3.6 | 90-100 | High | Good | Yes | Yes |
| PDMS | — | −123 | 0.22-0.22 | 1-5 | 5-8 | Low | Poor | Yes | Yes |
| (Silicon | | | | | | | | | |
| rubber) | | | | | | | | | |
| PC | 300 | 150 | 0.195-0.21 | 2-2.4 | 65-75 | Low | Medium | Yes | Yes |
| Austenitic | 1500 | — | 16-16 | 188 | 800-2200 | Medium | Poor | Yes | No |
| Stainless | | | | | | | | | |
| Steel: 316 | | | | | | | | | |
| LVM | | | | | | | | | |
| Titanium | 1650 | — | 7.2-7.2 | 115 | 1150 | High | Poor | Yes | No |
| Alloy | | | | | | | | | |

As shown in Table 3, acrylic polymer possesses good machinability, biocompatibility and also acceptable cost. Thus this material was chosen for development of initial prototypes. In alternative embodiments, the PEEK polymer can be employed although this polymer is a bit more costly.

### Selection of Micro-Milling Strategy and Parameters

The machining parameters used for the micro-milling process were selected starting with some recommended values and by improving them step by step. The most important parameters affecting the machining process were spindle speed, feed rate, axial depth of cut and air cooling system pressure. All these parameters are important to control the heat generation during the machining process. Heat generation is the most important phenomenon to be controlled because of very low thermal conductivity, glass transition temperature (Tg), and low melting point temperature (Tm) of these polymeric work materials. Machining process induces temperature increases which can be very close to glass transition temperature (even melting point temperature) creating low quality surface finish, chip melting and smearing onto the tool or the workpiece. Therefore machining parameters for micro-milling have to be optimized in order to avoid excessive temperature rises but also to maintain a desirable material removal rate. The feed rate was selected as 20 mm/min, the spindle speed was at 30000 rpm and the ADOC (for all the experiments) was taken as 0.1 mm. These values resulted in good surface finish during all the experiments. In addition, an air cooling system was introduced with 80 psi air pressure and a nozzle system directing at the cutting zone. This cooling system was introduced to avoid chip and burr melting problems.

### Fabrication and Experimental Set-Up

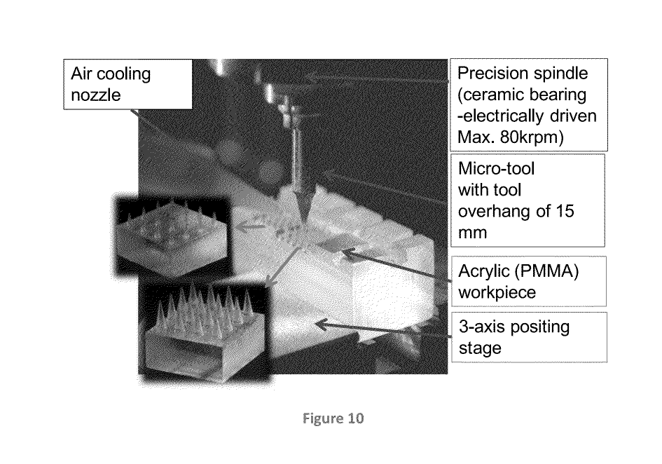

An experimental set-up for micro-milling of polymers shown in FIG. 10 has been prepared. In this experimental set-up, a 3-axis positioning stage driven by a computer numerical control system that accepts standard part programming (G codes) was utilized. A precision spindle (NSK ASTRO-E800) with ceramic bearings and electrically driven up to 80 krpm was employed. A micro-tool with a straight cutting edge was attached to the spindle using with a collet type to hold the overhanging about 18 mm. A rectangular polymeric workpiece was clamped on the fixture mounted on the table of this in-house developed micro-milling machine using a micro-precision vise. The workpiece surface was segmented by using a larger diameter flat endmill to the desired size for the micro-needle array base. In addition an air cooling system was introduced by using a nozzle to cool down the temperature in the polymer and also to air blow polymeric chips and debris.

### Tool-Path Strategies

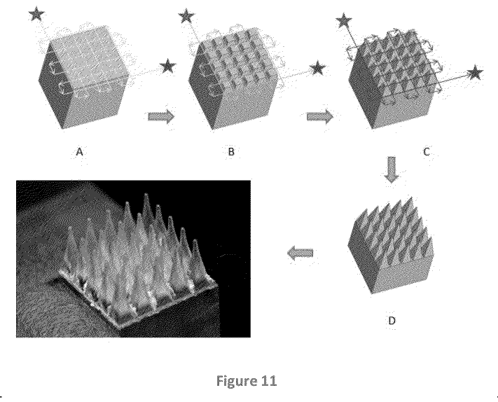

A solid model for the micro-needle arrays was developed to realized the overall geometry and some challenges in machining these geometries using micro-milling processes. Furthermore this model was utilized as a reference to generate the tool path and related NC part program. The first micro-needles array prototype has consisted of a pyramid shape with a square base. The tool path strategy that was utilized was a “Grill” type profile with a Z level increment of 0.1 mm [FIG. 11]. This strategy consisted of a zig-zag path with a stepover distance (essentially the distance between the center of micro-needles) on the longitudinal direction followed by the same zig-zag path in the transversal direction. The needles fabricated were 2 mm-tall, the base was a square with 0.7775 mm side length, and the micro-needle tips were 0.9075 mm apart from each other. In this case the array has 25 micro-needles arranged in 5 rows, and 5 columns. Therefore the workpiece has dimensions of 5.426×5.426×5 mm including the base (see FIG. 11). It is possible to see the different steps during the experiment and the resultant profiles, where the green star and the red one are the starting and the ending point on the same Z level tool path. The tool reaches to a deeper Z level as the color used for the tool path turns from yellow to red. This approach was found to be problematic after the experiment. The tips of the pyramid needles were not sharp enough because of bending occurred during the last several passes of the micro-milling operation. The situation was improved by modifying the tool path strategy and the micro-needle geometry.

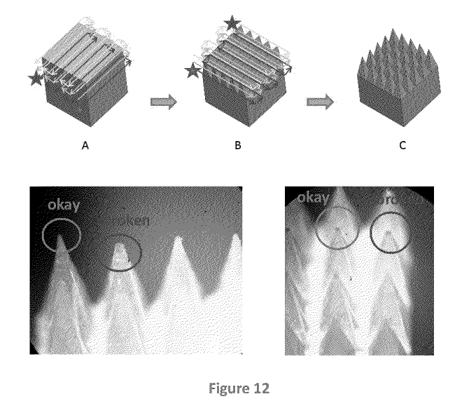

The second prototype was also a square base pyramidal shape micro-needle array but the tool path was different in order to avoid the aforementioned problem. This new tool path strategy was a “S” type tool path strategy. As shown in FIG. 12, the micro-needle tips were sharper but some of them were broken during the last few passes of the micro-milling operation. All the dimensions were same as in the first experiment.

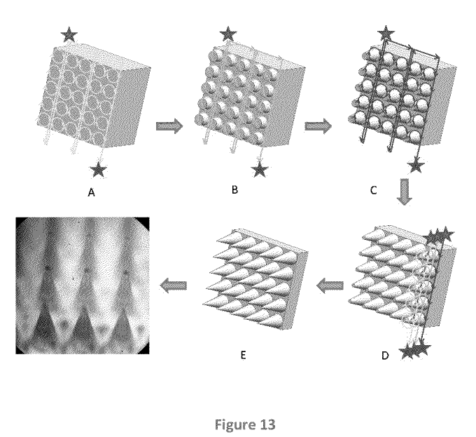

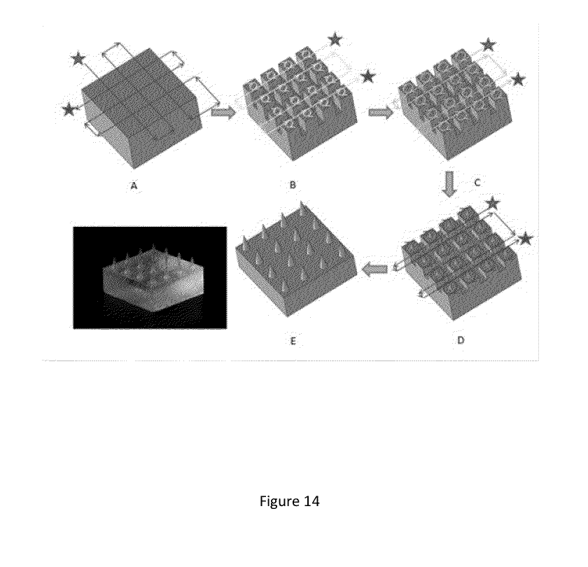

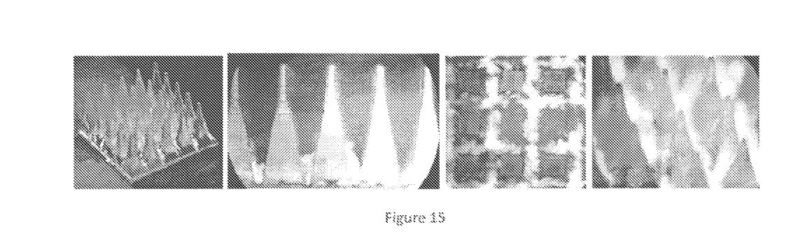

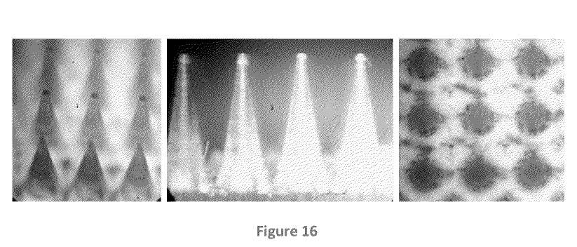

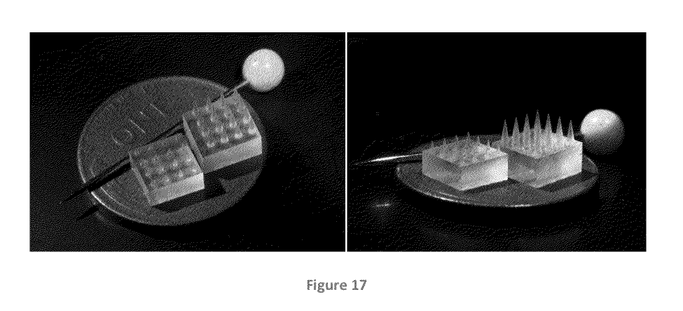

The third prototype provided a new shape for the needles and, related to this, a new tool path strategy was introduced. These micro-needles were cone-shaped and arranged in 5 rows and 5 columns amounting to 25 micro-needles on the array as shown in FIG. 13. The cone-shaped micro-needles were 2 mm-tall and the base radius was 0.382 mm. The workpiece had dimensions of 5.426×5.426×5 mm including the base. The Tool Path Strategy is shown in FIG. 14. A Z level increment of 0.1 mm was used. As it can be seen in FIG. 13 the cone-shaped micro-needles are sharper and well shaped than the pyramidal ones but also in this case micro-needle tips were deflected during the last few passes of the micro-milling operation.

In the fourth prototype a different tool path strategy was adopted. In this case the micro-needle array was designed with a larger space between each micro-needle and with shorter needles heights (see FIG. 14). This prototype has 16 micro-needles placed in 4 rows and 4 columns. The needles were only 1 mm tall in order to obtain higher stiffness and lower tip deflection. The base radius of micro-needles was 0.180 mm and the distance between the tips was 1.420 mm. The tool path strategy was changing at every Z level increment (0.1 mm). The idea was to start machining the tip of the needles in order to obtain sharper tips and not letting them to be tall and thin to avoid deflections. In the tool path, the radius of the circular movement to obtain the cones was increased at every Z level increment. Before starting with this step, a roughing operation was performed to avoid breaking tools and overheating/melting problems. The roughing operation was performed using a flat end-milling tool with a “Grill” tool path strategy (Step A, FIG. 14).

### Evaluation of the Results

As described above, these different prototypes result in different processing times. Beginning with the first prototype the machining times are increased due to longer tool path design. Particularly the last prototype required a tool change for roughing and finishing requiring more operator's time to perform it. The tool paths were improved to reduce the processing time required for completing the machining.

The cost is strictly related to the processing times needed to complete the machining operations. Since the work material used was the same in all experiments, the only variable affecting the cost was the number of tools used, tool life, the machine usage time, and the specialized operator's time needed.

As shown in FIG. 17, the quality of the prototype improved with each experiment, starting from the first attempt (a good shape was obtained but burrs and chip melted together), until the fourth prototype which is completely burrs-free and with the shape desired for the micro-needles.

Also the reliability of the fabrication process has been improved in the prototypes. The second prototype demonstrated some broken needles, but the needles were broken randomly during the micro-milling process making the fabrication process less reliable. The fourth micro-needle array was fabricated several times using the same micro-milling parameters and tool path strategy and the same results in dimensions, finishing and the shape were obtained consistently indicating that a reasonable reliability level was reached.

### CONCLUSIONS

Micro-needle arrays based patch prototypes were developed using micro-milling technology. Beginning with a simple idea, the prototype was improved in shape, fabrication strategy, dimensions and machining conditions in order to obtain the most satisfactory micro-needles patch prototype. The problems experienced during the experiments were mainly related to the tiny dimensions of the needles and the micro-machining of polymers, specially the heat generation problem. These problems were solved by modifying machining strategies and micro-milling process parameters. Using this strategy, hollow micro-needles comprising a reservoir for drug storage can be developed. The skilled person will appreciate that the needles shown in the figures are useful for “Transdermal Drug Delivery” of bioactive agents.

Where the bioactive agent is coated onto the micro-needle array, it may be advantageous to prepare the surface of the array before depositing a coating thereon. Useful methods of surface preparation can include, but are not limited to cleaning; physical modifications such as etching, drilling, cutting, or abrasion or roughing; and chemical modifications such as solvent treatment, the application of primer coatings, the application of surfactants, plasma treatment, ion bombardment, covalent bonding and electrochemical methods such as electropolishing, striking, electroplating and electrochemical deposition. Such surface preparation may serve to activate the surface and promote the deposition or adhesion of the coating on the surface. Surface preparation can also selectively alter the release rate of the bioactive.

Any additional coating layers can similarly be processed to promote the deposition or adhesion of another layer, to further control the release of the bioactive agent, or to otherwise improve the biocompatibility of the surface of the layers. For example, plasma treating an additional coating layer before depositing a bioactive agent thereon may improve the adhesion of the bioactive agent, increase the amount of bioactive agent that can be deposited, and allow the bioactive agent to be deposited in a more uniform manner.

A primer layer, or adhesion promotion layer, may also be applied to the micro-needle array. This layer may comprise, for example, silane, acrylate polymer/copolymer, acrylate carboxyl and/or hydroxyl copolymer, polyvinylpyrrolidone/vinylacetate copolymer (PVP/VA), olefin acrylic acid copolymer, ethylene acrylic acid copolymer, epoxy polymer, polyethylene glycol, polyethylene oxide, polyvinylpyridine copolymers, polyamide polymers/copolymers polyimide polymers/copolymers, ethylene vinylacetate copolymer and/or polyether sulfones.

The bioactive agent may be applied, for example, by spraying, dipping, pouring, pumping, brushing, wiping, vacuum deposition, vapor deposition, plasma deposition, electrostatic deposition, ultrasonic deposition, epitaxial growth, electrochemical deposition or any other method known to the skilled artisan. The bioactive agent may be applied as a separate layer or may be included in a layer also including a carrier material.

A variety of bioactive agents may be applied to the micro-needle array in accordance with the intended use. For example, antithrombogenic agents may be applied to the array. An antithrombogenic agent is any agent that inhibits or prevents thrombus formation within a body vessel. Types of antithrombotic agents include anticoagulants, antiplatelets, and fibrinolytics. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic agents include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole; fibrinolytics such as alfimeprase, alteplase, anistreplase, reteplase, lanoteplase, monteplase, tenecteplase, urokinase, streptokinase, or phospholipid encapsulated microbubbles; and other bioactive agents such as endothelial progenitor cells or endothelial cells.

Other bioactive agents that may be applied include antiproliferative/antimitotic agents including natural products such as vinca alkaloids (vinblastine, vincristine, and vinorelbine), paclitaxel, rapamycin analogs, epidipodophyllotoxins (etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (for example, L-asparaginase which systemically metabolizes L-asparagine and deprives cells which do not have the capacity to synthesize their own asparagine); antiplatelet agents such as (GP) IIb/IIIa inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat.

In a preferred embodiment, the bioactive agent applied to the micro-needle array is selected from the group consisting of paclitaxel, rapamycin, a rapamycin derivative, an antisense oligonucleotide, an siRNA, and a mTOR inhibitor.

### REFERENCES

[1] S Henry, D V McAllister, M G Allen and M R Prausnitz, Microfabricated Microneedles: A Novel Approach to Transdermal Drug Delivery, Journal of Pharmaceutical Sciences, 1998, 87: 922-925.
[2] A. Maton; J. Hopkins, C. W. McLaughlin, S. Johnson, M. Q. Warner, D. LaHart, J. D. Wright (1993). Human Biology and Health. Englewood Cliffs, N.J., USA: Prentice Hall.
[3] Georgia Tech Research News, Microneedles: report describes progress in developing new technology for painless drug vaccine delivery, Nov. 17, 2003.
[4] J. Zachary Hilt, Nicholas A. Peppas, Microfabricated drug delivery devices, International Journal of Pharmaceutics 306 (2005) 15-23
[5] S. Hashmi, P. Ling, G. Hashmi, M. Reed, R. Gaugler, W. Trimmer, Genetic transformation of nematodes using arrays of micromechanical piercing structures, BioTechniques 19(1995) 766-770.
[6] S. Kaushik, A. H. Hord, D. D. Denson, D. V. McAllister, S. Smitra, M. G. Allen, M. R. Prausnitz, Lack of pain associated with microfabricated microneedles, Anesth. Analg. 92 (2001) 502-504.
[7] J. A. Mikszta, J. B. Alarcon, J. M. Brittingham, D. E. Sutter, R. J. Pettis, N. G. Harvey, Improved genetic immunization via micromechanical disruption of skin-barrier function and targeted epidermal delivery, Nat. Med. 8 (2002) 415-419.
[8] W. H. Smart, K. Subramanian, The use of silicon microfabrication technology in painless blood glucose monitoring, Diabetes Technol. Ther. 2 (2000) 549-559.
[9] Micro-Needles for Drug Delivery, Vaccines, and Medical Diagnostics, yet2.com Inc., tow0039236.pdf 10 Dec. 2006.
[10] J. H. Park, M. G. Allen, M. R. Prausnitz, J. Control. Release 104 (2005) 51-66.
[11] Jeong W. Lee, Jung-Hwan Park, Mark R. Prausnitz, Dissolving microneedles for transdermal drug delivery. Biomaterials 29 (2008) 2113-2124
[12] Harvinder S. Gill, Mark R. Prausnitz, Coated microneedles for transdermal delivery. Journal of Controlled Release 117 (2007) 227-237
[13] S. C. Kuo, Y. Chou. A Novel Polymer Microneedle Arrays and PDMS Micromolding Technique. Tamkang Journal of Science and Engineering, Vol. 7, No. 2, (2004) pp. 95-98

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A micro-milled patch comprising a plurality of micro-needles, said micro-needles comprising a structure comprising a base at a proximal end and a vertex or tip, defining a vertex angle, at a distal end, said needle not extending into the dermis when applied to the skin, wherein said micro-needle is optionally biodegradable.

2. The patch of claim 1, wherein said micro-needles are fabricated using a biodegradable polymer.

3. The patch of claim 2, wherein said micro-needles comprise a bio-active agent.

4. The patch of claim 3, wherein said bioactive agent is a vaccine.

5. The patch of claim 1, wherein said micro-needle comprises a conical configuration.

6. The patch of claim 1, wherein said micro-needle comprises a pyramidal configuration.

7. The patch of claim 1, wherein said micro-needle comprises a cylindrical configuration.

8. A single flute straight cutting edge tungsten carbide engraving tool for fabrication of micro-needles having the geometrical parameters of Table 2.

9. A method for delivering a bioactive agent to a patient in need thereof comprising applying the patch of claim 1 to the skin of said patient such that a bioactive agent gains access to the blood stream of said patient wherein said micro-needle does not penetrate the dermis of said patient.

10. The method of claim 9, wherein said micro-needles are biodegradable.

11. The method of claim 10, wherein said bioactive agent is a vaccine.

12. A method of manufacturing the patch comprising the plurality of micro-needle structures of claim 1 comprising the steps of: providing a suitable material from which said patch and structures can be fabricated by means of one or more micro-milling techniques; fabricating said patch and structures from said suitable material by means of one or more micro-milling techniques, wherein said micro-needle structures have a proximal end defining a base and a distal end having a vertex wherein said base has a diameter in the range from about 0.3 to about 0.7 mm and the line extending from the center of the base to the vertex defines a structural axis having a length in the range from about 0.6 to 1.5 mm, wherein the distal end intersects said vertex; and customizing a tip at said vertex end, said customized tip being selectively angled for a particular application.

13. The method according to claim 12, wherein said manufacturing is performed using a micro-milling process and the micro-tool of claim 8.

14. A kit for delivering a bioactive agent to the skin of a patient in need thereof, the kit comprising the patch of claim 1 and instructional materials for use thereof.

15. A structure comprising a plurality of micro-needles for delivering a formulation to across a biological barrier, comprising: at least one micro-needle according to claim 1 configured to penetrate the biological barrier; and a reservoir in fluid communication with said at least one micro-needle, wherein said reservoir is configured to contain a volume of formulation.
